(19) United States
(12) Patent Application Publication
(10) Pub. No.: US 2007/0129350 A1
(43) Pub. Date: Jun. 7, 2007

(54) Methods for treatment of diabetes

(75) Inventor: Gosse B. Bruinsma, Leiden (NL)

Correspondence Address: TRASK BRITT, P.O. BOX 2550, SALT LAKE CITY, UT, 84110, US

(73) Assignee: Axonyx, Inc., New York City, NY (US)

(21) Appl. No.: 10/587,578

(22) PCT Filed: Jan. 28, 2005
(86) PCT No.: PCT/US05/02559

§ 371 Date: Jul. 28, 2006

Related U.S. Application Data
(60) Provisional application No. 60/540,722, filed on Jan. 30, 2004.

(51) Int. Cl. A61K 31/55; A61K 31/473; A61K 31/445; A61K 31/407

(52) U.S. Cl. 514/214.01; 514/411; 514/319; 514/297

(57) ABSTRACT

Phenserine compounds and pharmaceutically acceptable salts thereof are found to be useful in the management or treatment of diabetes and/or complications, such as vascular dementia, and insulin resistance, associated with diabetes. The phenserine compounds of the invention are carbamates that inhibit the activity of acetylcholinesterase.

### CROSS-REFERENCE TO RELATED APPLICATIONS

This application claims the benefit under 35 U.S.C. § 119(e) of U.S. Provisional Application No. 60/540,722, filed Jan. 30, 2004, the entirety of which is incorporated by this reference.

### TECHNICAL FIELD

The invention relates to pharmaceutical compounds generally and, more particularly, to various methods and compositions for the treatment of diabetes and associated sequelae.

### BACKGROUND

Physostigmines, also called “eserines,” are known cholinesterase inhibitors. These compounds are also useful in the treatment of glaucoma, Myasthenia Gravis, and Alzheimer's disease, and as antidotes against poisoning with organophosphates.

The natural isomer of physostigmine has blocking properties, as well as, agonist properties at the neuromuscular acetylcholine receptor (AChR). By contrast, (+)-physostigmine shows only negligible inhibition of cholinesterase (ChE). See Brossi et al., FEBS Lett., Vol 201, pages 190-192 (1986).

Even though (+)-physostigmine has only negligible ChE inhibitory activity, it is effective as a protective pretreatment drug against multiple lethal doses of sarin, see Albuquerque et al, Fundam. Appl. Caltoxicol., Vol. 5, pages 182-203 (1985). The observed beneficial protection appears to be due to direct interactions of the carbamates with the postsynaptic nicotinic ACHR. The protective effectiveness of the carbamates against organophosphates appears to be related to the direct ability of the carbamates to decrease the hyperactivation caused by accumulation of the neurotransmitter.

Diabetes mellitus affects about 17 million citizens of the United States and is the 5^{th} leading cause of death by disease in the United States. Direct and indirect medical expenditures attributable to diabetes were estimated at 132 billion US dollars (see Hogan et al., (2003) Economic costs of diabetes in the US in 2002, Diabetes Care, 26(3):917-932). Direct medical expenditures alone totaled 91.8 billion US dollars and comprised 23.2 billion US dollars for diabetes care, 24.6 billion US dollars for chronic complications attributable to diabetes, and 44.1 billion US dollars for excess prevalence of general medical conditions (Id.). Hogan et al. report that in 2002, diabetes more than doubled the cost of health care in the United States.

Thus, diabetes imposes a substantial economic burden to society and, in particular, to those individuals with diabetes and their families. Hogan et al. stated that “[e]liminating or reducing the health problems caused by diabetes through factors such as better access to preventive care, more widespread diagnosis, more intensive disease management, and the advent of new medical technologies could significantly improve the quality of life for people with diabetes and their families while at the same time potentially reducing national expenditures for health care services and increasing productivity in the U.S. economy” (Id. at 917). Therefore, a long felt need exists for improved disease management, particularly, new and improved treatments.

Diabetes mellitus is also considered a risk factor for the development of vascular dementia, such as Alzheimer's disease (AD). Insulin's role as a neuromodulator in the brain is beginning to emerge, raising a question regarding its significance for AD. Insulin dysregulation may contribute to AD pathology through several mechanisms including decreased cortical glucose utilization particularly in the hippocampus and entorhinal cortex; increased oxidative stress through the formation of advanced glycation end-products; increased Tau phosphorylation and neurofibrillary tangle formation; and increased β-amyloid (Aβ) aggregation through inhibition of insulin-degrading enzyme. Thus, effective treatment of diabetes mellitus may also prevent or slow the onset of diabetes associated sequelae.

It has been reported that erythrocyte membrane protein glycosylation increases by 3.4 fold in diabetes (Dave, Patel, Katyare, (2001) Indian J. Clinical Biochem., 16(1):81-88). However, insulin or sulfonylurea treatment was not reported to reduce the extent of glycosylation (Id.). These authors also reported that erythrocyte membrane acetylcholinesterase activity decreased only in the sulfonylurea treated group (Id.). In particular, the Vmax of acetylcholinesterase decreased only in the sulfonylurea treated group (Id.). While, serum butyrylcholinesterase activity was relatively low in the diabetic and insulin treated diabetic groups, the diabetic state exhibited a decreased Vmax for components I and II of serum butyrylcholinesterase (Id.). Further, these authors report that in vitro incubation with insulin differentially affected the Na plus, K plus-ATPase and serum butyrylcholinesterase activities (Id.).

In addition, erythrocyte membrane acetylcholinesterase activity has been reported to be significantly decreased in insulin-dependent diabetes mellitus, with activity negatively correlating with the fasting blood glucose level (see Suhail and Rizvi, (1989), Erythrocyte membrane acetylcholinesterase in type 1 (insulin-dependent) diabetes mellitus, Biochem. J., 259:897-899). This report suggested that the number of active enzyme molecules (AChE) in diabetes was reduced.

Previous studies have also demonstrated that PKC-dependent processes are involved in both ACH-induced Ca2+-signaling and insulin secretion. For example, the neurotransmitter acetylcholine (ACh) increases cytosolic free calcium and is thought to stimulate insulin secretion from pancreatic beta-cells by activating receptors coupled to phosphatidylinositol breakdown, thereby, generating IP3 and diacylglycerol, which activates protein kinase C (PKC).

Finally, it has been reported that ACh plays a role in the release of hepatic insulin sensitizing substance and the treatment of insulin resistance. This report stated that administration of ACh by a non-liver specific mechanism (intravenous) did not reverse insulin resistance induced by surgical denerveation.

Thus, the literature provides indefinite and conflicting reports of potential links between acetylcholine, acetylcholinesterase and diabetes, including types I and II.

Accordingly, a need in the art exists for selective agents, active in vivo, having an acceptable therapeutic window, and minimal side effects, for treating diabetes mellitus and associated complications.

### DISCLOSURE OF INVENTION

The invention relates to a method of treating a subject comprising administering an effective amount of a phenserine compound or phenserine-like compound of the invention or an effective amount of a pharmaceutical composition according to the invention to a subject, e.g., a mammal, such as a human, thought to be in need of such treatment.

The invention also relates to a method of treating diabetes mellitus and/or associated sequelae, such as reducing the risk of vascular dementia associated therewith or delaying the onset of such a complication, comprising treating a subject with an effective amount of a phenserine compound, for example, phenserine, ((−)-N-phenylcarbamoyl eseroline), and/or the (+) isomer of phenserine, and/or a pharmaceutically acceptable salt or ester thereof. A pharmaceutically acceptable salt is preferably a tartrate, a phosphate, or a fumarate salt.

The invention also relates to a method of treating diabetes mellitus and/or associated sequelae by treating a subject with a phenserine-like compound, such as donepezil, galantamine, rivastigme and/or tacrine. Phenserine and/or phenserine-like compounds may be used to prevent or reduce insulin resistance and/or to treat dementia associated with Aβ protein and neurofibrillary tangles.

The invention also relates to pharmaceutical compositions comprising an effective amount of a phenserine compound and/or phenserine-like compound and/or a pharmaceutically acceptable salt or ester thereof, and a method for the treatment of diabetes mellitus associated sequelae and/or the risk of vascular dementia.

The invention also relates to pharmaceutical compositions comprising an effective amount of a phenserine compound and/or phenserine-like compound and/or a pharmaceutically acceptable salt or ester thereof, and a method for the treatment of diabetes mellitus associated sequelae and/or insulin resistance.

The invention also relates to a method according to the invention comprising administering to the subject an effective amount of a phenserine compound and/or phenserine-like compound of the invention or a pharmaceutical composition according to the invention, in combination with a hypoglycemic agent selected from the group consisting of sulfonylureas, meglitinides, biguanides, thiazolidinediones, alpha-glucosidase inhibitors, and/or mixtures thereof.

The invention also relates to a method of treating a diabetic condition or complication, for example, a subject's blood glucose levels (hyperglycaemia or hypoglycaemia), carbohydrate intake levels, responsiveness or non-responsiveness to hypoglycemic agents, diabetic neuropathy, diabetic retinopathy, vascular dementia, kidney function, pregnancy, ketone levels, hyperlipidaemia, and/or coronary artery disease, by administering to the subject an effective amount of a phenserine compound and/or phenserine-like compound of the invention or a pharmaceutical composition according to the invention.

The invention further relates to a use of a pharmaceutical compound of the invention in the manufacture of a medicament for the treatment of diabetes and/or diabetes associated sequelae. The invention also relates to the manufacture of a pharmaceutical composition comprising a phenserine compound and/or a phenserine-like compound and/or a pharmaceutically acceptable salt thereof for the treatment of diabetes mellitus and/or associated sequelae, such as the risk of vascular dementia and/or insulin resistance.

### BEST MODES FOR CARRYING OUT THE INVENTION

Surprisingly, the phenserine compounds and phenserine-like compounds according to the present invention are useful in the management, treatment and/or prevention of diabetes and/or complications associated with diabetes. The phenserine compounds and other compounds of the invention produce fewer undesirable side effects than other carbamate analogues known in the art. Further, the phenserine compounds are more brain-targeted versus the rest of the body and are more rapidly cleared from the blood than other AChEIs. Accordingly, the method for treating diseases, such as diabetes and/or associated sequelae (e.g., vascular dementia, insulin resistance, hyperglycemia, responsiveness or non-responsiveness to hypoglycemic agents, and/or diabetic neuropathy) using compounds according to the present invention represent a significant advance over the prior art.

Diabetes mellitus is a disease in which the body does not produce and/or properly use insulin. Improper use of insulin (e.g., insulin resistance) is one of the underlying causes of type II diabetes and may lead to type I diabetes if the pancreatic cells fail due to the insulin secretion demand placed on them. Insulin resistance occurs when the body fails to respond properly to the insulin it already produces. Ninety percent of people with type II diabetes are thought to be insulin resistant to some extent. Furthermore, insulin resistance may affect more than 60 million Americans, with one in four of them likely to develop type II diabetes. Additionally, research indicates that insulin resistance is associated with an increased risk for heart disease and stroke.

Insulin is a hormone that is needed to convert sugar, starches and other food into energy needed for daily life. The most common forms of diabetes are type I (also referred to as “insulin dependent diabetes” or “juvenile diabetes”) or type II diabetes (also referred to as “non-insulin dependent diabetes” or “adult on-set diabetes”), although, other classifications exists, for example, diabetes bronze, which typically results from pancreatic damage caused by iron deposition, and gestational diabetes, which typically appears during pregnancy and disappears after birth.

Currently, there are five distinct classes of hypoglycemic agents available for the treatment of type II diabetes, each class displaying unique pharmacologic properties. These classes are the sulfonylureas, meglitinides, biguanides, thiazolidinediones, and alpha-glucosidase inhibitors. The invention provides another class of agents, (e.g., phenserine compounds and/or phenserine-like compounds), useful in the treatment of diabetes, such as type II diabetes. The phenserine compounds and/or phenserine-like compounds may be administered alone or in combination with one or more hypoglycemic agents.

Acetylcholinesterase inhibitors, such as the phenserine and phenserine-like compounds, may be useful in the treatment of insulin resistance. However, insulin resistance develops in the liver, therefore, it may be presumed to be preferable to minimize the diffusion of the acetylcholine esterase agonist into the spinal cord and brain. In particular, administration of ACh targeted to the liver is a logical choice, as opposed to the brain and spinal cord. Thus, the development of insulin resistance in the liver teaches away from the use of the compounds of the present invention, as the compounds (e.g., phenserine) are more brain-targeted versus the rest of the body and are more rapidly cleared from the blood than other AChEIs.

As used herein, “treatment of diabetes” and the “management of diabetes,” are used interchangeably and does not necessarily mean a complete cure. It means that the symptoms or complications of the underlying disease are reduced, and/or that one or more of the underlying cellular, physiological, or biochemical causes or mechanisms causing the symptoms or complications are reduced. It is understood that “reduced,” as used in this context, means relative to the untreated state of the disease, including the molecular state of the disease, not just the physiological state of the disease. The term treatment of diabetes also includes within its scope the prophylactic treatment of an asymptomatic subject, such as a mammal, particularly a human, thought to be at risk of developing diabetes.

As used herein, “effective amount” means an amount of an active ingredient administered to the patient, which will be effective to improve, prevent, delay the onset of, or treat the disease condition or associated complications in the patient.

Phenserine, (−)-N-phenyl carbamoyleseroline, has the structure:

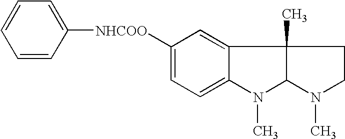

Phenserine, ((−)-N-phenylcarbamoyl eseroline), is a carbamate analog of physostigmine (Phy), which is a long-acting inhibitor of cholinesterase. Phenserine was first prepared by Polonovski, (1916), Bull. Soc. Chim. 19, 46-59, and technical details were summarized by Beilstein, Handbuch der Organischen Chemie, 4th edn. vol 23. Springer Verlag, Berlin, pp 333 (1954)). It was reported in the literature without any stated practical use.

In addition, the phenserine compounds of the present invention include the (+) isomer of phenserine, which has the following structure: 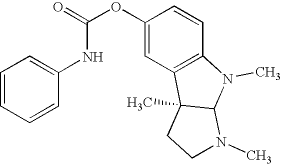
(3aS)-1,3a,8-trimethyl-1,2,3,3a,8,8a-hexahydropyrrolo[2,3-b]indol-5-yl phenylcarbamate

The phenserine compounds of the invention may be synthesized, for example, by using processes known in the art. For example, U.S. Pat. Nos. 6,495,700, 5,409,948, 5,171,750, 5,378,723, and 5,998,460, and International Patent Publication WO 03/082270 A1, all of which are hereby incorporated by reference in their entirety, describe the preparation of phenserine compounds of the invention and assays that may be used to test compounds of the invention. The phenserine compounds of the invention include carbamates having specificity for the inhibition of acetylcholinesterase and/or inhibition of β-AAP synthesis, including, but not limited to, (−)-N-phenylcarbamoyl eseroline (which may also be referred to as (3aR)-1,3a,8-trimethyl-1,2,3,3a,8,8a-hexahydropyrrolo[2,3-b]indol-5-yl phenylcarbamate or phenserine); (3aS)-1,3a,8-trimethyl-1,2,3,3a,8,8a-hexahydropyrrolo[2,3-b]indol-5-yl phenylcarbamate or Posiphen™; (−)-2′-methylphenylcarbamoyleseroline; (−)-2′4′dimethylphenylcarbamoyleseroline; (−)-4′-methylphenylarbamoyleseroline; (−)-2′-ethylphenylcarbamoyleseroline; (−)-phenylcarbamoyleserolne; (−)-(−)-2′,4′,6′trimethylphenylcarbamoyleseroline; (−)-2′-chlorophenylcarbamoyleseroline; (−)-2′,6′-dichlorophenylcarbamoylseroline; (−)-physovenol; (−)-5-O-(2′-methylphenylcarbamoyl)physovenol; (−)-3,3a,8,8a-tetrahydro-3a,8-dimethyl-2H-thieno-[2,3-b]indole-5-ol butyl carbamate; (−)-3,3a,8,8a-tetrahydro-3a,8-dimethyl-2H-thieno[2,3-b]indole-5-ol heptylcarbamate; (−)-3,3a,8,8a-tetrahydro-3a,8-dimethyl-2H-thieno[2,3-b]indole-5-ol phenylcarbamate; (−)-3,3a,8,8a-tetrahydro-3a,8-dimethyl;-2H-thieno[2,3-b]indole-5-ol 2′-methylphenylcarbamate; (−)-3,3a,8,8a-tetrahydro-3a,8-dimethy-2H-thieno[2,3-b]indole-5-ol 2′-isopropylphenylcarbamate; (−)-thiaphysovenine, (−)-Phenyl-thiaphysovenine; (−)-2′,4′-dimethylphenyl-thiaphysovenine and/or pharmaceutically acceptable salts thereof. Additional compounds that may be used in the invention include (3aS)-3a-methyl-1,2,3,3a,8,8a-hexahydropyrrol[2,3-b]indol-5-yl N-4′-isopropylphenylcarbamate; (3aR)-3a-methyl-1,2,3,3a,8,8a-hexahydropyrrol[2,3-b]indol-5-yl N-4′-isopropylphenylcarbamate.

The phenserine-like compounds of the invention include functionally related compounds, such as donepezil, galantamine, rivastigme and/or tacrine. These compounds may be used to treat diabetes, including prevention or reduction in insulin resistance and/or to treat dementia associated with Aβ protein and neurofibrillary tangles. In addition, other AChE inhibitors are known in the art and may also be used according to the methods of the present invention.

Salts, esters and the free base of the compounds of the invention are within the scope of the present invention and are included by reference to one or more compound. Hence, reference to phenserine or donepezil includes pharmaceutically acceptable salts and/or esters thereof. Pharmaceutically acceptable salts are known in the art, for example, salts of phenserine, and include, but are not limited to, tartrate, phosphate, and fumarate salts.

Potential cholinesterase agents can be evaluated for potency in vitro by testing the agents against electric eel and human red blood cell acetylcholinesterase (AChE) and human plasma butyrylcholinesterase, (BChE) (see also, U.S. Patents: U.S. Pat. Nos. 6,495,700; 5,409,948; 5,171,750; 5,378,723; and 5,998,460).

The effect of the cholinesterase agents of the invention may be tested for their potency in the reduction of insulin resistance using methods known in the art, for example, as described in U.S. Pat. No. 5,561,165, RIST, ITT and the HIEC tests (see, Reid et al., (2002), Comparison of the rapid insulin sensitivity test (RIST), the insulin tolerance test (ITT), and the hyperinsulinemic euglycemic clamp (HIEC) to measure insulin action in rats, Can. J. Physiol. Pharmacol. 80:811-818). Subjects thought to have insulin resistance may be tested using methods known in the art, for example, by the glucose tolerance test, three hour glucose tolerance test, or other tests known in the art.

The phenserine and phenserine-like compounds of the invention are also useful in the treatment of vascular dementia. In an exemplary embodiment, one or more phenserine compound and/or phenserine-like compound is used to treat the presence and/or accumulation of the Aβ protein associated with vascular dementia and/or neurofibrillary tangles.

Two substrates of insulin-degrading enzyme (IDE), amyloid β-protein (Aβ) and insulin, are critically important in the pathogenesis of Alzheimer's Disease and type II diabetes mellitus, respectively. IDE has been identified as a principal regulator of Aβ levels in neuronal and microglial cells and a mutant IDE allele has been associated with hyperinsulinemia and glucose intolerance in a rat model of type II diabetes (Farris et al. (2002) Insulin-degrading enzyme regulates the levels of insulin, amyloid β-protein, and the β-amyloid precursor protein intracellular domain in vivo, Proc. Natl. Acad. Sci. USA 100(7):4162-4167). Human genetic studies have implicated the IDE region of chromosome 10 in both AD and type II diabetes. Id.

Type II diabetes may be a risk factor for dementia, but the associated pathological mechanisms remain unclear. However, diabetes is increasingly associated with total dementia, Alzheimer's disease, and vascular dementia. Individuals with both type II diabetes and the APOE epsilon4 allele have nearly a doubled risk for AD compared with those with neither risk factor. Subjects with type II diabetes and the epsilon4 allele have a higher number of hippocampal neuritic plaques and neurofibrillary tangles in the cortex and hippocampus, and they have a higher risk of cerebral amyloid angiopathy. Thus, the association between diabetes and AD is particularly strong among carriers of the APOE epsilon4 allele (Peila et al., (2002) Type 2 diabetes, APOE gene, and the risk for dementia and related pathologies: The Honolulu-Asia Aging Study, Diabetes 51(4):1256-62). The present invention provides methods of treating diabetes, for example, insulin resistance and/or neurological conditions associated with diabetes.

Compositions within the scope of the invention include compositions wherein the active ingredient is contained in an effective amount to achieve its intended purpose. Effective concentrations may range from 0.001 wt % to 1.0 wt %. The compounds can be administered in any pharmaceutically acceptable amount, for example, in amounts ranging from 0.001 gram to about 1 gram per kilogram of body weight. Based on the information which is presented herein, the determination of effective amounts is well within the skill of the ordinary practitioner in the art. In addition, the ordinary practitioner may formulate the dosage regimen as appropriate for the diabetic condition being treated. For example, the compositions of the invention may be administered orally prior to carbohydrate intake, at times of hypoglycemia or hyperglycemia. Where a compound of the invention is administered prior to carbohydrate intake, the compound may be administered about 3 times a day.

The compounds are generally used in pharmaceutical compositions (wt %) containing the active ingredient with a carrier, vehicle, diluent and/or excipient in the composition in an amount of about 0.1 to 99 wt % and preferably about 25-85 wt %. Pharmaceutical compositions may be formulated using carriers, diluents and/or excipients known in the art, for example, see REMINGTON'S PHARMACEUTICAL SCIENCES, 18th Ed. (1990, Mack Publishing Co., Easton, Pa.).

The compounds may be administered in any desired form, including parenterally, orally, injection, transdermally or by suppository using known methods.

Either fluid or solid unit dosage forms can be readily prepared for oral administration. For example, the active compounds can be admixed with conventional ingredients such as dicalcium phosphate, magnesium aluminum silicate, magnesium stearate, calcium sulfate, starch, talc, lactose, acacia, methyl cellulose and functionally similar materials as pharmaceutical excipients or carriers.

A sustained release formulation may optionally be used where appropriate or desirable. Capsules may be formulated by mixing the compound with a pharmaceutical diluent which is inert and inserting this mixture into a hard gelatin capsule having the appropriate size. If soft capsules are desired, a slurry of the compound with an acceptable vegetable, light petroleum or other inert oil can be encapsulated by forming into a gelatin capsule.

Suspensions, syrups and elixirs may be used for oral administration of fluid unit dosage forms. A fluid preparation including oil may be used for oil soluble forms. A vegetable oil such as corn oil, peanut oil or sunflower oil, for example, together with flavoring agents, sweeteners and any preservatives produces an acceptable fluid preparation. A surfactant may be added to water to form a syrup for fluid unit dosages. Hydro-alcoholic pharmaceutical preparations may be used having an acceptable sweetener (such as sugar, saccharin, or a biological sweetener, preferably a low carbohydrate sweetener, such as manitol or sorbitol) and a flavoring agent in the form of an elixir.

Pharmaceutical compositions for parenteral and suppository administration can also be obtained using techniques standard in the art. In an exemplary embodiment, the compounds of the invention are administered as pharmaceutical agents suitable for oral administration. In another exemplary embodiment, the compounds of the invention may be administered by injection in an appropriate vehicle such as sesame oil.

The pharmaceutical carriers acceptable for the purposes of this invention include all art recognized carriers that do not exhibit a significant adverse affect on the drug, the host, or the material comprising the drug delivery device or vehicle. Suitable pharmaceutical carriers include sterile water, saline, sorbitol, sucralose, manitol, manitol in water or saline condensation products of castor oil and ethylene oxide combining about 30 to 35 moles of ethylene oxide per mole of castor oil, liquid acid, lower alkanols, oils such as corn oil, peanut oil, sesame oil and the like, with emulsifiers such as mono- or di-glyceride of a fatty acid; or a phosphatide, e.g., lecithin, and the like; glycols, polyalkylene glycols, aqueous media in the presence of a suspending agent, for example, sodium carboxymethyl cellulose, sodium alginate, poly(vinylpyrrolidone), and the like, alone, or with suitable dispensing agents such as lecithin, polyoxyethylene stearate, and the like. The carrier may also contain adjuvants such as preserving agents, stabilizing agents, wetting agents, emulsifying agents and the like together with penetration enhancer and the compounds of this invention.

The effective dose for mammals may vary due to such factors as age, weight, activity level or condition of the subject being treated. Typically, an effective dosage of a compound according to the present invention is about 1 to 800 milligrams when administered by either oral or rectal dose from 1 to 3 times daily. This is about 0.002 to about 50 milligrams per kilogram of the subject's weight administered per day. Preferably about 10 to about 300 milligrams are administered orally or rectally 1 to 3 times a day for an adult human. The required dose is usually considerably less when administered parenterally. Preferably about 0.01 to about 150 milligrams may be administered intramuscularly, one to three times a day for an adult human.

In an exemplary embodiment, the method according to the invention comprises administering an effective amount of a phenserine compound and/or phenserine-like compound of the invention and/or an effective amount of a pharmaceutical composition according to the invention to a subject, such as a mammal, thought to be in need of such treatment. For example, a subject that may benefit from the present invention is a subject suffering from insulin resistance, diabetes and/or vascular dementia. In another exemplary embodiment, the method according to the invention comprises administering to a subject an effective amount of a phenserine compound and/or phenserine-like compound of the invention and/or a pharmaceutical composition according to the invention, in combination with a hypoglycemic agent selected from the group consisting of sulfonylureas, meglitinides, biguanides, thiazolidinediones, alpha-glucosidase inhibitors or mixtures thereof. In yet another exemplary embodiment, the invention provides a method of preparing a pharmaceutical useful in the treatment of diabetes and/or vascular dementia.

In another exemplary embodiment, the invention provides a prophylactic treatment, for example, a prophylactic treatment to reduce the risk of or delay the onset of gestational diabetes or to treat insulin resistance prior to the onset of diabetes. Treatment of insulin resistance prior to the onset of diabetes, or prior to the onset of type I diabetes in a type II diabetic patient, may be particularly advantageous in elderly subjects. Elderly subjects may be experiencing cognitive decline and may potentially experience more than one benefit, for example, the cognitive improvement from the cholinergic effects of a phenserine compound and/or phenserine-like compound, a decrease in insulin resistance, and/or decreased accumulation of Aβ. Elderly subjects include humans greater than about 45 years of age, greater than about 50 years of age, greater than about 55 years of age, greater than about 60 years of age, greater than about 65 years of age, greater than about 70 years of age, greater than about 75 years of age, and greater than about 80 years of age.

In an exemplary embodiment, a phenserine compound and/or phenserine-like compound is administered in combination with an increase in insulin levels. For example, the phenserine compound and/or phenserine-like compound may be administered in combination with a bolus of insulin, either an insulin injection or the action of an agent which stimulates the release of insulin. In another exemplary embodiment, the phenserine compound and/or phenserine-like compound is administered prior to each meal.

As will be recognized by a person of ordinary skill in the art, treatment of diabetes, such as type I or II diabetes, is affected by numerous conditions. For example, the subject's blood glucose levels (hyperglycaemia or hypoglycaemia), carbohydrate intake levels, response to hypoglycemic agents, diabetic neuropathy, diabetic retinopathy, vascular dementia, kidney function, pregnancy, ketone levels, hyperlipidaemia, and coronary artery disease.

Mice useful in the study of type I diabetes may be obtained, for example, from The Jackson Laboratory Type 1 Diabetes Repository (T1DR) (stocks are available online at jax.org/t1dr/holdings.html). Protocols for the study of diabetes using mouse models are known in the art, for example, as described in Leiter, E. H. Current Protocols in Immunology §§ 15.9.1-15.9.23 (John Wiley & Sons, Inc. eds. 1997). A mouse model for type II diabetes has been described by Fernandez et al., (2001) Functional inactivation of the IGF-I and insulin receptors in skeletal muscle causes type 2 diabetes, Genes Dev. 15(15):1926-34.

Without wishing to be bound by any theory, the phenserine compounds and/or phenserine-like compounds of the invention are believed to inhibit acetylcholinesterase activity, increasing acetylcholine levels, thereby effecting utilization of insulin by a subject. In addition, compounds of the invention reduce neurofibrillary tangle formation and decrease β-amyloid aggregation, thereby reducing the risk of developing vascular dementia (e.g., cerebral amyloid angiopathy), which is associated with diabetes.

The presence of insulin in the blood elicits a hepatic parasympathetic reflex, stimulating release of ACh in the liver. The release of ACh releases nitric oxide, which acts to control the sensitivity of skeletal muscle to insulin through the action of a liver released hormone, the hepatic insulin sensitizing substance (HISS). HISS selectively stimulates glucose uptake and storage as glycogen in tissues including skeletal muscle. In the absence of HISS, muscle cells are resistant to insulin and insulin driven storage of glucose by skeletal muscle is reduced.

HISS release in response to insulin is affected by the fasting state of the subject. Specifically, in the fasting state HISS release is minimal and insulin produces a minimal metabolic effect. Following a meal, the parasympathetic reflex mechanism is amplified, allowing release of HISS and more efficient utilization of insulin for the storage of glucose in skeletal muscle.

Decreased release of HISS may result in severe insulin resistance, which may be referred to as HISS-dependent insulin resistance (“HDIR”). In the absence of HISS, the pancreas is required to secrete substantially larger amounts of insulin to compensate for the resistance. Persistent insulin resistance is a leading cause of type II diabetes (non-insulin dependent diabetes mellitus) and may lead to a complete exhaustion of the pancreas, thus requiring the patient to resort to insulin injections.

The phenserine compounds and/or phenserine-like compounds of the invention provide the ability to reduce insulin resistance, thereby providing a treatment for diabetes. Further, the compounds of the invention may be used to prevent cognitive disorders frequently associated with diabetes or reduce the risk of vascular dementia.

### EXAMPLE I

Experimental Protocol for Determining Effect on Insulin Resistance:

Test animals, for example, non-obese diabetic mice, are anesthetized and an arterial-venous shunt is introduced into the animal according to procedures known in the art. The arterial-venous shunt allows for blood sampling and infusion of test compounds.

Baseline blood glucose levels are established following surgery. Insulin is then introduced into the animal and glucose infused so as to maintain a steady glucose level throughout the period of insulin activity. By measurement of the glucose infusion rate throughout the experiment, the effect of the insulin is measured.

The compounds are tested by introducing the test compound at the appropriate time relative to the insulin administration and measuring the effect on glucose infusion. The compounds of the invention increase the rate of glucose infusion relative to control animals.

For example, phenserine administered approximately 30 minutes prior to the administration of insulin is found to increase the rate of glucose infusion. Thus, a phenserine compound and/or phenserine-like compound is found to reduce insulin resistance and increases the effectiveness of the administered insulin.

### EXAMPLE II

Blood glucose levels are established in fasting subjects. A predetermined dose of insulin is then administered to the patients followed by feeding the patients meal having a set carbohydrate content. Blood glucose levels are monitored before, during and for at least 4 hours following administration of the insulin.

The patients are subsequently fasted and the experiment repeated with administration of the test compound prior to administration of the insulin. Comparison of the blood glucose levels for the subjects with and without the test compound is performed to determine the effect of the test compound on insulin utilization by the patient.

Care is taken to avoid hypoglycemic episodes and occurrence of a hypoglycemic episode, in a subject which would not normally experience such an episode, may be taken as evidence of the compounds effect on insulin resistance.

### EXAMPLE III

β-APP synthesis may be measured in vitro or in vivo by methods known in the art. For example, by an ELISA assay or a Western. The test compound may be administered to a subject for in vivo testing and β-APP levels assayed at various time points.

Alternatively, cells may be cultured in the presence of a pulse of a labeled amino acid, the label washed off, and the test compound applied to the cells. Label incorporated into the β-APP protein is then quantitated to determine the effect of the compound on the synthesis of β-APP.

The effect of the test compound on protein synthesis or protein stability may also be determined by other methods known in the art.

All references, including publications, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method for treating diabetes in a subject, said method comprising:
admixing at least one phenserine compound or phenserine-like compound, and salts or esters thereof, and a pharmaceutically acceptable diluent, carrier or excipient to form a medicament; and
administering the medicament to the subject, thereby treating diabetes in said subject.

2. The method according to claim 1, wherein the phenserine compound is selected from the group consisting of (−)-N-phenylcarbamoyl eseroline, (3aS)-1,3a,8-trimethyl-1,2,3,3a,8,8a-hexahydropyrrolo[2,3-b]indol-5-yl phenylcarbamate; (−)-2′-methylphenylcarbamoyleseroline, (−)-2′-4′-dimethylphenylcarbamoyleseroline, (−)-4′methylphenylarbamoyleseroline, (−)-2′-ethylphenylcarbamoyleseroline, (−)-phenylcarbamoyleserolne, (−)-(−)-2′,4′,6′-trimethylphenylcarbamoyleseroline, (−)-2′-chlorophenylcarbamoyleseroline, (−)-2′,6′-dichlorophenylcarbamoylseroline, (−)-physovenol, (−)-5-O-(2′-methylphenylcarbamoyl)physovenol, (−)-3,3a,8,8a-tetrahydro-3a,8-dimethyl-2H-thieno-[2,3-b]indole-5-ol butyl carbamate, (−)-3,3a,8,8a-tetrahydro-3a,8-dimethyl-2H-thieno[2,3-b]indole-5-ol heptylcarbamate, (−)-3,3a,8,8a-tetrahydro-3a,8-dimethyl-2H-thieno[2,3-b]indole-5-ol phenylcarbamate, (−)-3,3a,8,8a-tetrahydro-3a,8-dimethyl-2H-thieno[2,3-b]indole-5-ol 2′-methylphenylcarbamate, (−)-3,3a,8,8a-tetrahydro-3a,8-dimethy-2H-thieno[2,3-b]indole-5-ol 2′-isopropylphenylcarbamate, (−)-thiaphysovenine, (−)-Phenylthiaphysovenine, and (−)-2′,4′-dimethylphenyl-thiaphysovenine.

3. The method according to claim 2, wherein the phenserine-like compound is selected from the group consisting of donepezil, galantamine, rivastigme and tacrine.

4. The method according to claim 2, wherein the phenserine compound comprises (−)-N-phenylcarbamoyl eseroline or a salt or ester thereof.

5. The method according to claim 1, wherein treating diabetes comprises delaying onset of a cognitive impairment associated with diabetes.

6. The method according to claim 1, wherein treating diabetes comprises treating gestational diabetes.

7. The method according to claim 1, wherein treating diabetes comprises a prophylactic treatment of diabetes.

8. The method according to claim 1, wherein treating diabetes comprises treating cerebral angiopathy.

9. The method according to claim 1, wherein the subject is a human above the age of 60 years.

10. The method according to claim 1, further comprising administering said phenserine compound or phenserine-like compound prior to a meal.

11. The method according to claim 10, further comprising coadministering a hypoglycemic agent selected from the group consisting of a sulfonylurea, a meglitinide, a biguanide, a thiazolidinedione, an alpha-glucosidase inhibitor and mixtures thereof.

12. The method according to claim 1, wherein treating diabetes comprises treating at least one condition selected from the group consisting of hyperglycaemia, hypoglycaemia, response to hypoglycemic agents, diabetic neuropathy, diabetic retinopathy, kidney function, vascular dementia, ketone levels, hyperlipidaemia, and coronary artery disease.

13. The method according to claim 1, wherein treating diabetes comprises treating insulin resistance.

14.-18. (canceled)
